(19) United States
(12) Patent Application Publication
(10) Pub. No.: US 2021/0342713 A1
(43) Pub. Date: Nov. 4, 2021

(54) ENVIRONMENTAL AND CROP MONITORING SYSTEM

(71) Applicant: Bioverse Labs Corp, Plantation, FL (US)

(72) Inventors: Francisco D'Elia, Plantation, FL (US); Christos Stamatopoulous, Plantation, FL (US); Dylan Riffle, Weston, FL (US)

(73) Assignee: Bioverse Labs Corp, Plantation, FL (US)

(21) Appl. No.: 16/866,367

(22) Filed: May 4, 2020

(51) Int. Cl. G06N 5/04; G01D 21/02; G01N 33/00; G06N 20/00; A01M 1/02; A01M 1/14

(52) U.S. Cl. CPC G06N 5/04; G01D 21/02; A01M 1/14; G06N 20/00; A01M 1/026; G01N 33/0098

(57) ABSTRACT

An environmental and crop monitoring system is disclosed, comprising a plurality of sensors disposed in an environment. The plurality of sensors is configured to dynamically detect environmental anomalies (e.g., within crops) and transmit output data to a processing system in communication with the plurality of sensors. The processing system is configured to predict the anomalies associate with environmental or crop monitoring.

### CROSS-REFERENCE TO RELATED APPLICATIONS

The present application claims priority to U.S. Provisional Patent Application 62/842,780 filed on May 4, 2019, entitled “ENVIRONMENTAL AND CROP MONITORING SYSTEM” the entire disclosure of which is incorporated by reference herein.

### TECHNICAL FIELD

The embodiments generally relate to systems for monitoring, classifying, and analyzing species and biological communities within environments including agriculture.

### BACKGROUND

The agricultural industry relies on healthy crops to maximize profits. There are a number of variables which impact crop production including environmental conditions, the quantity and quality of nutrients in the environment, plant condition, and the presence of invasive and beneficial species. Additionally, invasive species and population monitoring are required for the evaluation of environmental health conditions.

Modern agricultural practices include monitoring field health and acting in response to data gathered from such monitoring to improve field and crop growth efficiency. The process of monitoring crop health is often time and labor intensive, relying on human systems to accurately and thoroughly analyze a high volume of crops.

Annual losses in food production due to invasive species alone is estimated to have reached $1.4 Trillion globally. In the current arts, solutions to the presence of invasive species include the introduction of pesticides or other mitigating factors. The agricultural industry continually seeks ways to detect the presence of invasive species as early as possible to reduce a loss in the total yield of crops, as well as promoting important species that provide ecosystems services such as pollination.

### SUMMARY OF THE INVENTION

This summary is provided to introduce a variety of concepts in a simplified form that is further disclosed in the detailed description of the embodiments. This summary is not intended to identify key or essential inventive concepts of the claimed subject matter, nor is it intended for determining the scope of the claimed subject matter.

The embodiments described herein provide for an environmental monitoring system including crop monitoring capabilities comprising a plurality of sensors disposed in an environment. The plurality of sensors is configured to dynamically detect crop and environmental anomalies and transmit output data to a processing system in communication with the plurality of sensors. The processing system is configured to predict the crop and environmental anomalies.

The sensor array may be provided on an insect trap having an adhesive surface to capture the insect and retain the insect thereon. The sensor array may capture imagery of the insect and transmit the imagery to a machine learning module to compare the imagery with imagery stored in a database to identify the insect. In such, the system may determine the species of the insect and if the insect is a detrimental or beneficial to the crops. Similarly, the machine learning system may be utilized to determine crop infections, crop nutrient deficiencies, etc.

In one aspect, the plurality of sensors is configured to move throughout the environment via a sensor positioning system.

In another aspect, the sensor positioning system is comprised of at least one of the following: a cabling system, a rail system, a magnetic line system, optical sensors, audio sensors and air quality sensors.

In one aspect, the sensor positioning system is further comprised of one or more UAV's configured to move at least one sensor throughout the environment.

In one aspect, the plurality of sensors is comprised of at least one of the following: a GNSS system, an optical camera, an RGBD camera, a thermal camera, a hyperspectral camera, a humidity sensor, a temperature sensor, a pressure sensor, or a luminosity sensor, a CO2 sensor and microphones.

In one aspect, the plurality of sensors transmits output data to a database. The database is in operable communication with an artificial intelligence engine configured to identify biological community and predict environmental and crop anomalies.

### BRIEF DESCRIPTION OF THE DRAWINGS

A complete understanding of the present invention and the advantages and features thereof will be more readily understood by reference to the following detailed description when considered in conjunction with the accompanying drawings wherein:

FIG. 1 illustrates a block diagram of the data capture process, according to some embodiments;

FIG. 2 illustrates a schematic of the crop monitoring system including an automated crop scanner, a sensor package, a plant condition analysis interface, and a hotspot detection interface, according to some embodiments;

FIG. 3 illustrates a schematic of the smart sensor trap system, according to some embodiments;

FIG. 4 illustrates a schematic of the modular smart sensor system, according to some embodiments;

FIG. 5 illustrates a block diagram of the data collection process and 3D positioning system, according to some embodiments;

FIG. 6 illustrates perspective views of the sensor positioning systems including a cable system, a rail system, and a magnetic line system, according to some embodiments;

FIG. 7 illustrates a computer-generated image of the graphical user interface for detecting plant stressors and pathogens, according to some embodiments;

FIG. 8 illustrates a flowchart for the processes of data input, analysis, reinforcement learning, and visualization of model results, according to some embodiments;

FIG. 9 illustrates a side elevation view of the insect trap and sensor according to some embodiments;

FIG. 10 illustrates a perspective view of the insect trap and sensor according to some embodiments;

FIG. 11 illustrates a flowchart of the data and machine learning system, according to some embodiments;

FIG. 12 illustrates a screenshot of the species monitoring system, according to some embodiments; and

FIG. 13 illustrates a schematic of the dataflow and machine learning system, according to some embodiments.

### DETAILED DESCRIPTION

The specific details of the single embodiment or variety of embodiments described herein are to the described system. Any specific details of the embodiments are used for demonstration purposes only and no unnecessary limitations or inferences are to be understood therefrom.

Before describing in detail exemplary embodiments, it is noted that the embodiments reside primarily in combinations of components related to the systems described herein. In general, the embodiments relate to systems and methods for monitoring, analyzing, and treating an agricultural environment. The agricultural environment may include a large environment such as an entire outdoor crop field, or indoor or semi-indoor greenhouse, or be as small as a single plant.

In some embodiments, a crop monitoring system collects data from a plurality of sensors positioned in a sensor array in an environment. The plurality of sensors may be static or may be capable of moving throughout the environment. In one example, at least a portion of the plurality of sensors are engaged with a fixed cable system to facilitate movement of the portion of the plurality of sensors throughout the environment to autonomously scan the environment for anomalies.

The crop monitoring system may operate autonomously without requiring user intervention. The system may instruct one or more of the plurality of sensors to move to the desired location, collect data, and migrate to a subsequent location.

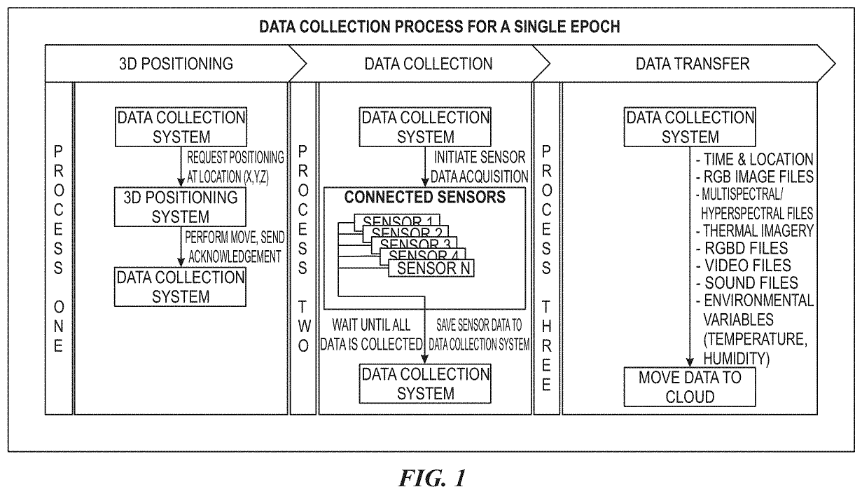

In some embodiments, and in reference to FIG. 1, data collection can include the capture and digitization of information using the plurality of sensors. The data collected may include microscopy images, terrestrial and close-range imagery, aerial imagery, satellite imagery, real color (RGB) imagery, multi-spectral imagery, hyperspectral imagery, audio data, and thermal imagery. In further embodiments, data can include geolocation data, date, time, and technical details of each type of imagery captured. Data is collected and aggregated, labeled, and processed for storage in a database.

The plurality of sensors may also include global navigation satellite systems (GNSS), optical cameras, RGBD cameras, thermal cameras, hyperspectral cameras, humidity sensors, temperature sensors, pressure sensors, and luminosity sensors.

In some embodiments, the plurality of sensors is comprised of at least one hyperspectral camera and at least one RGB camera to facilitate the detection of plant anomalies, such as adverse responses to environmental stressors. In one example, the plurality of sensors detects changes in chlorophyll production (NDVI reflectance) and transpiration (temperature). The system may then correlate detected changes to a causative agent.

In some embodiments, the crop monitoring system is comprised of an alert system such that upon the detection of an anomaly, an alert is transmitted to notify agricultural personnel of the potential of a problem with the environment or crops therein.

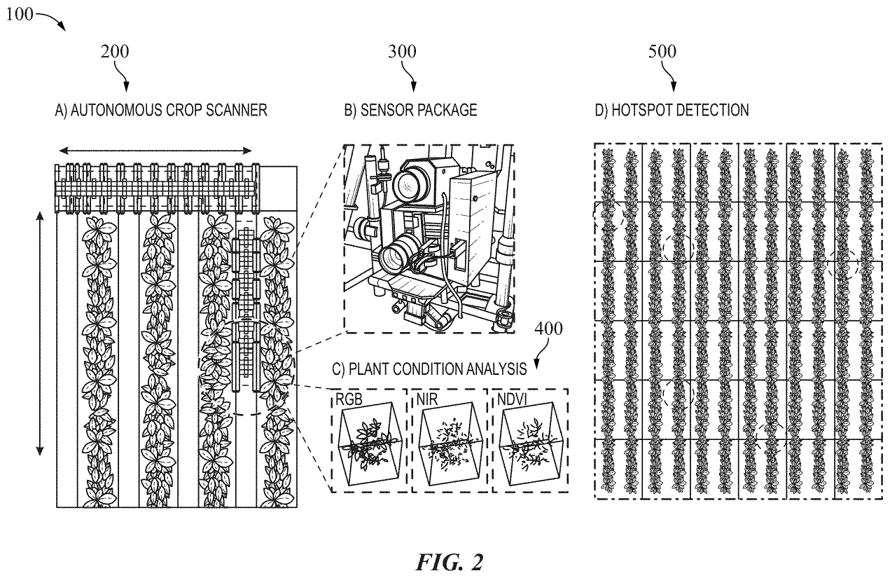

FIG. 2 illustrates a crop monitoring system 100 comprised of an autonomous crop scanner 200, a sensor package 300, a plant condition analysis interface 400 and a hotspot detection interface 500.

In some embodiments, the crop monitoring system 100 is comprised of a database to store historical data related to the environment and crops therein. The system 100 can generate models to predict hotspots before they are detected by the plurality of sensors.

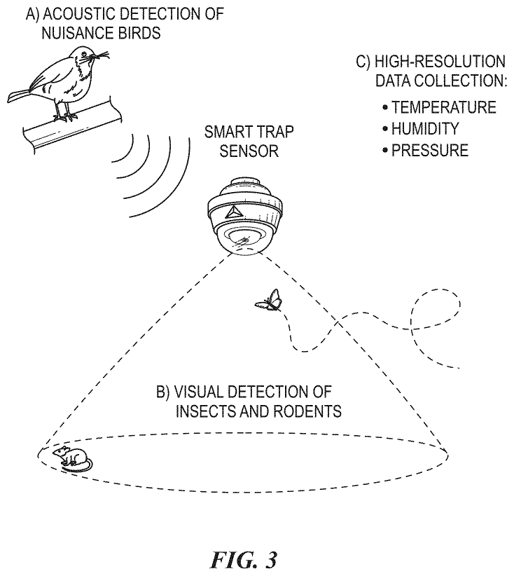

In reference to FIG. 3, a smart trap sensor is configured to perform real-time surveillance of crops in an environment by collecting acoustic and imagery data to accurately identify species using on-board algorithms. Additionally, the sensors collect environmental data such as temperature, humidity, pressure and CO2 to generate predictive models in tandem with the crop monitoring system. In some embodiments, multiple smart trap sensors are configured to form a network throughout the environment. The smart trap sensors are configured to be modular and can be arranged in varying densities to suit specific environmental organization schemes.

In some embodiments, the smart trap sensors may be placed external to the environment to detect the presence of pests which may enter the environment.

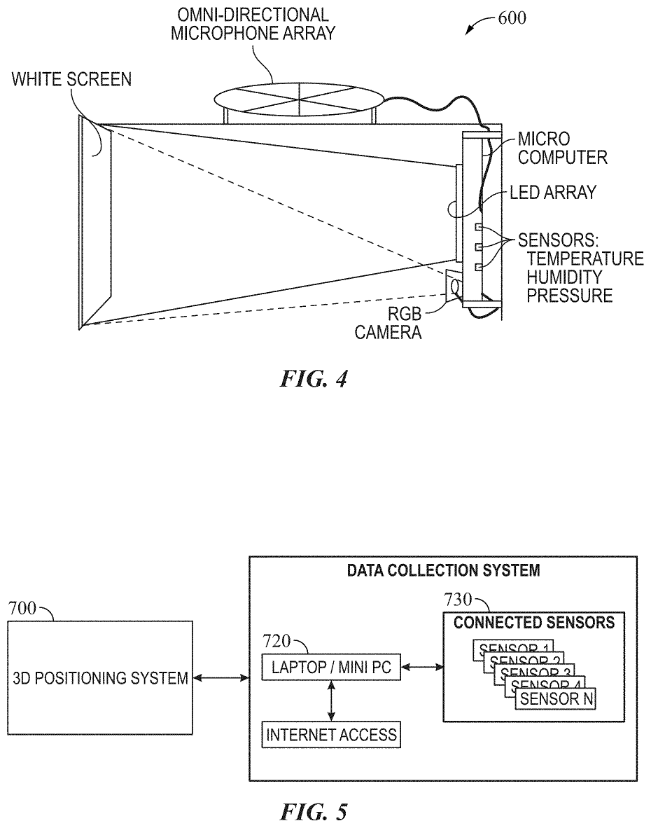

FIG. 4 illustrates a schematic of the modular smart sensor array 600 configured to capture environmental and crop data using a plurality of sensors in an array. The plurality of sensors may include any combination of the plurality of sensors described hereinabove.

FIG. 5 illustrates a block diagram of the crop monitoring system including a 3D positioning system 700, and a computing device 720 in communication with the plurality of sensors 730. The plurality of sensors may move freely in the environment to collect the necessary environmental and crop data for post-processing by the crop monitoring system.

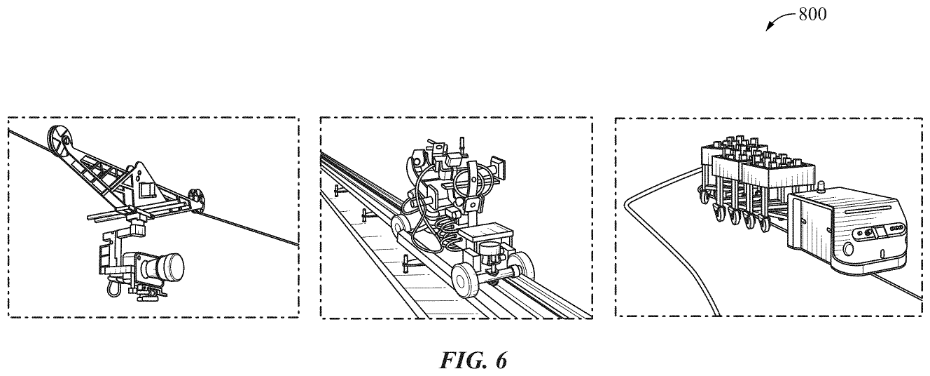

FIG. 6 illustrates exemplary images of the sensor positioning system 800 comprised of cabling systems, rail systems, and magnetic line systems. Further, the sensor positioning system may utilize drones, UAV technology, or similar forms of sensor mobility implements. In some examples, at least a portion of the plurality of sensors are affixed to a gimbal to reduce vibration while permitting rotation and articulation of the affixed sensors. Aerial positioning systems can include multi-rotor UAV's, larger fixed-wing UAV's, and fixed-wing planes and/or helicopters.

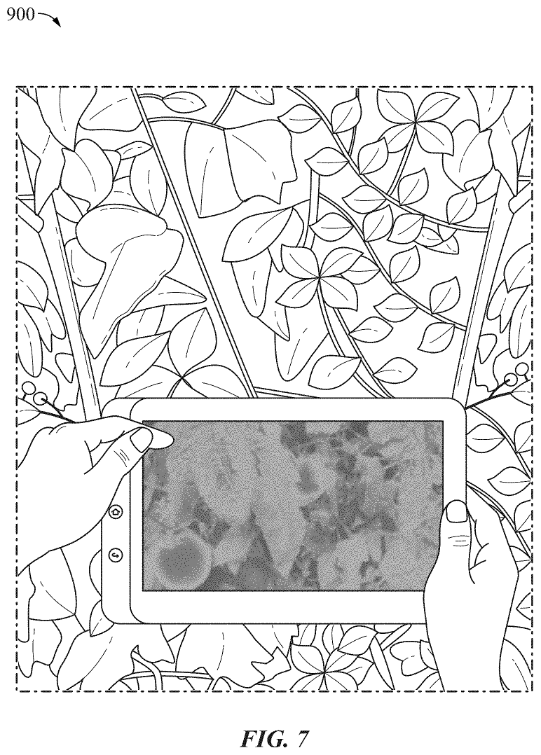

FIG. 7 illustrates a user interface for a mobile application 900 comprised of an augmented reality engine configured to detect crop anomalies. The mobile application 900 may be in communication with the crop monitoring system to determine crop anomalies and display the crop anomalies on a graphical user interface of a computing device. The mobile application 900 improves the efficiency of human scouts in detecting pests, pathogens, or plant stressors through the use of augmented reality software to aid in the visualization of plant anomalies which are otherwise difficult for a human to detect.

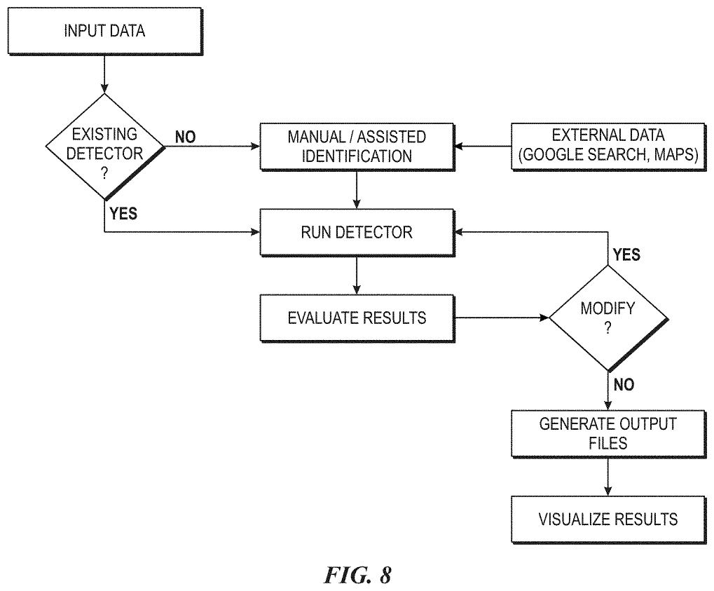

In some embodiments, the crop monitoring system is in communication with a species identification system configured to aid a user in producing semantic or thematic maps using artificial intelligence algorithms. The system also allows for the identification of biological organisms in the environment. FIG. 8 illustrates a flowchart of the data input, analysis, reinforcement learning, and visualization of model results.

Content and/or data interacted with, requested, or edited in association with one or computing devices may be stored in different communication channels or other storage types. For example, data may be stored using a directory service, a web portal, a mailbox service, an instant messaging store, or a compiled networking service for managing preloaded and/or updated maps of agricultural fields or similar environments. A computing device may provide a request to a cloud/network, which is then processed by a server in communication with an external data provider. By way of example, a client computing device may be implemented as any of the systems described herein and embodied in a personal computing device, a tablet computing device, and/or a mobile computing device (e.g., a smartphone). Any of these aspects of the systems described herein may obtain content from the external data provider.

In various embodiments, the types of networks used for communication between the computing devices that make up the present invention include, but are not limited to, an internet, an intranet, wide area networks (WAN), local area networks (LAN), virtual private networks (VPN), radio communication devices, cellular networks, and additional satellite-based data providers such as the Iridium satellite constellation which provides voice and data coverage to satellite phones, pagers and integrated transceivers, etc. According to aspects of the present disclosure, the networks may include an enterprise network and a network through which a client computing device may access an enterprise network. According to additional aspects, a client network is a separate network accessing an enterprise network through externally available entry points, such as a gateway, a remote access protocol, or a public or private Internet address.

Additionally, the logical operations may be implemented as algorithms in software, firmware, analog/digital circuitry, and/or any combination thereof, without deviating from the scope of the present disclosure. The software, firmware, or similar sequence of computer instructions may be encoded and stored upon a computer readable storage medium. The software, firmware, or similar sequence of computer instructions may also be encoded within a carrier-wave signal for transmission between computing devices.

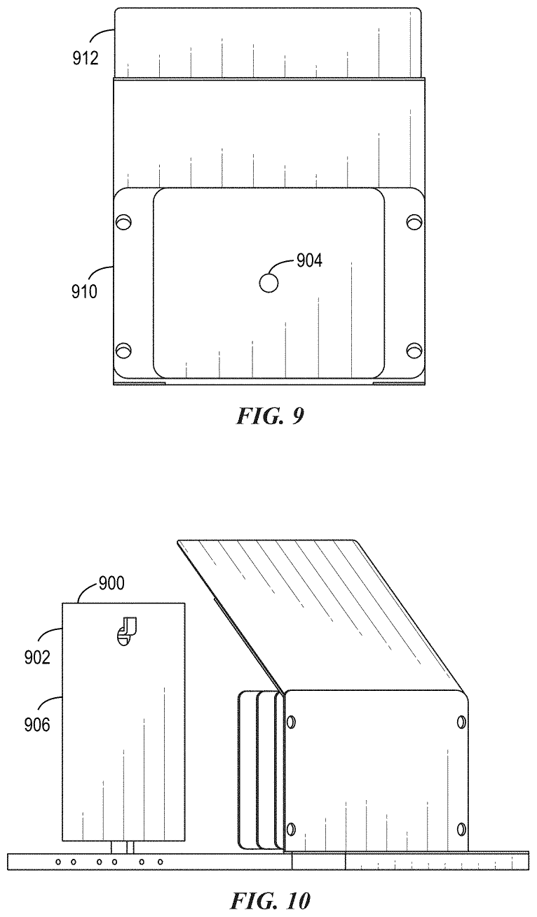

FIG. 9 and FIG. 10 illustrate an insect trap 900 having a trap component 902 and a sensor array 904 to capture information related to an insect which is retained by the trap component 902. The trap component may include a surface 906 having an adhesive provided thereon. The surface 906 may be baited to attract the insect before ensnaring the insect onto the surface. The adhesive may include any adhesive substance known in the arts. In some embodiments, the trap component 902 may be a sheltered trap to take advantage of an insect's tendency to seek shelter in certain environmental mediums such as loose bark, crevices, or other sheltered environment mediums. The sensor array 904 may include one or more cameras to capture an image of trapped insects. The imagery may be transmitted to the environmental and crop monitoring systems described herein. The imagery may be processed by a machine learning module to identify the type and species of the insect, such as by using a comparator to compare known stored images of insects with the imagery transmitted by the sensor array. A computational module may be provided behind a front casing 910 to create data contextualizing the imagery the sensor collects.

In some embodiments, the insect trap 900 and sensor array 904 thereof may include a solar cell in communication with a solar panel mounting to the front casing of the trap 200 to increase the autonomy of the device by eliminating the need to charge the device.

In some embodiments, the insect trap 900 and sensor array 904 may be at least partially water resistant using cables glands, silicon paste, and water right connections between the components permits the sensor array 904 to be deployed in rain, wind, snow, and other potentially hazardous conditions.

In some embodiments, the sensor array 904 may include a sensing module in operable communication with the computing module to gather information related to environmental conditions, humidity, CO2 levels, temperature, and the like.

In some embodiments, the sensor array 904 and sensing module may be configured to capture environmental audio information to analyze bioacoustics of the environment. In one example, the sensor array 904 is comprised of one or more microphones positioned behind the sensor casing to generate audio files of environmental sounds in real-time. The audio files may be uploaded to the environmental and crop monitoring system to contextualize the data via converting the audio files to stereographs. Machine learning models may be used to analyze and identify the sources which created the sounds provided on the audio files.

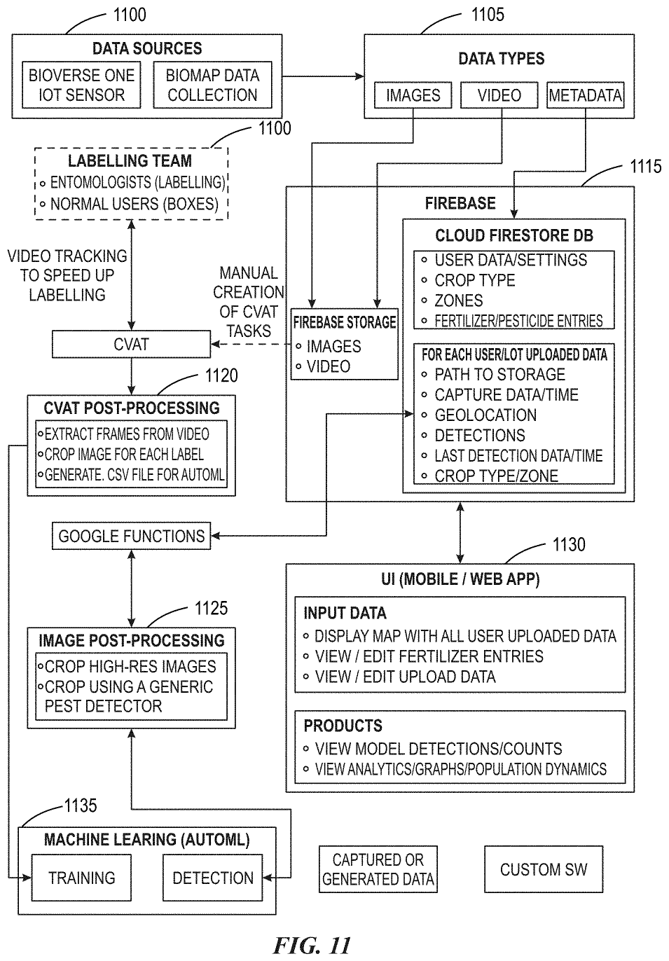

FIG. 11 illustrates a block diagram of the information system including data resources 1100, data type 1105, labelling 1110, storage elements 1115, processors 1120, image processors 1125, user interface engines 1130, and machine learning engines 1135 to operate the various functionalities described herein.

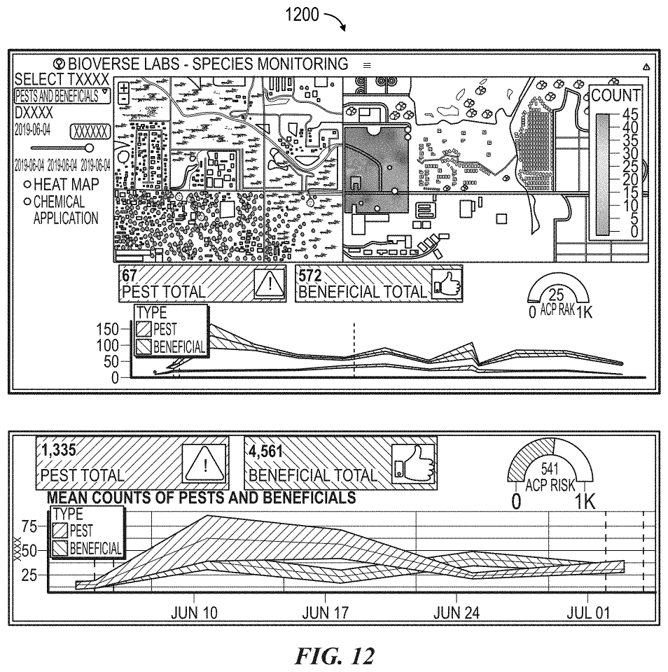

FIG. 12 illustrates an analytics dashboard 1200 comprising biodiversity reports, clients alerts, client customization settings, and similar information. The analytics dashboard is configured to assist the customers organization or users to assign other users to assist on filed data collection tasks, allowing the creation of a team to perform various crop monitoring functions. Biodiversity reports employ spatially distributed heat maps of biodiversity, real-time graphs, and meters such that the client receives accurate reports on the biodiversity of their property. Client alerts permit the client to establish alerts for when populations reach a certain threshold which instructs sensors to go online or offline. The alerts may be programmed to alarm when environmental pests or contaminants appear, as well as alarm when various environmental events occur. The client may use the user interface to customize their dashboard to display various environmental factors or likewise information.

In some embodiments, the environmental and crop monitoring systems described herein is operable on a computing device having an application system downloaded thereon to execute the various functionalities of the system. The application system may store imagery captured by the sensor array or the camera associated with the users mobile computing device. The application system employs a user interface to aggregate user and organization information to contextualize the collected environmental information.

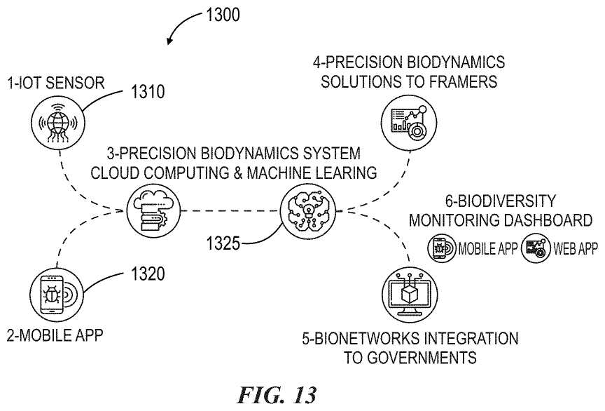

FIG. 13 illustrates the precision biodynamics system 1300 comprising one or more IoT sensors 1310 to aggregate information related to the crops and their environments. A mobile application 1320 provides a user interface to customize the display of information, interact with information, and otherwise engage with the system as described hereinabove. The machine learning module 1325 implements insect and plant identification processes by comparing receives information from the sensors and comparing the received information with stored reference information. A biodynamics solution is provided to farmers, agriculturalists, and the like to provide an analytics dashboard.

The system is provided for the is digitization, automation, and is demonetization of invasive species monitoring services. Our solutions have applicability in outdoor and indoor farming worldwide. Our solution helps farmers to reduce the use of pesticides, risk of crop loss by invasive species or diseases, and reduce impact in the environment while reducing the operational costs of pest management, it opens possibilities to promote pollinators, natural predators and more sustainable agricultural systems, such as organic and biodynamic farming.

Data APIs are generated from the collection of sensors and devices of a given region. The focus is on providing detailed information to government agencies, academia and private sector about trends in crop condition, insect population, climate condition and forecasts based on machine learning models or deep learning models developed to query the data based on a given area of concern.

Many different embodiments have been disclosed herein, in connection with the above description and the drawings. It will be understood that it would be unduly repetitious and obfuscating to literally describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

An equivalent substitution of two or more elements can be made for any one of the elements in the claims below or that a single element can be substituted for two or more elements in a claim. Although elements can be described above as acting in certain combinations and even initially claimed as such, it is to be expressly understood that one or more elements from a claimed combination can in some cases be excised from the combination and that the claimed combination can be directed to a subcombination or variation of a subcombination.

It will be appreciated by persons skilled in the art that the present embodiment is not limited to what has been particularly shown and described hereinabove. A variety of modifications and variations are possible in light of the above teachings without departing from the following claims.

What is claimed is:

1. An environmental monitoring system, comprising:
a plurality of sensors disposed in an environment, the plurality of sensors configured to dynamically detect crop and environmental anomalies;
a processing system in communication with the plurality of sensors, the processing system configured to predict environmental anomalies;
a machine learning engine to receive environmental information from a sensor array and compare, via a comparator, the received information with information stored in a database to identify the environmental information.

2. The system of claim 1, wherein the plurality of sensors are provided in a housing of an insect trap.

3. The system of claim 2, wherein the insect trap includes an adhesive surface to retain the insect on the insect trap.

4. The system of claim 1, wherein the environmental information is comprised of crop species, and insect species.

5. The system of claim 1, wherein the plurality of sensors are configured to move throughout the environment via a sensor positioning system.

6. The system of claim 5, wherein the sensor positioning system is comprised of at least one of the following:
a cabling system;
a rail system;
a magnetic line system; or
a fixed post

7. The system of claim 6, wherein the sensor positioning system is further comprised of one or more UAV's configured to move at least one sensor throughout the environment.

8. The system of claim 1, wherein the plurality of sensors is comprised of at least one of the following:
a GNSS system;
an optical camera;
an RGBD camera;
a thermal camera;
a hyperspectral camera;
a humidity sensor;
a temperature sensor;
a pressure sensor;
a luminosity sensor;
a CO2 sensor; or
an audio sensor.

9. The system of claim 8, wherein the plurality of sensors transmit output data to a database.

10. The system of claim 9, wherein the database is in operable communication with an artificial intelligence engine configured to predict environmental and crop anomalies.

11. A modular smart sensor system, comprising:
a plurality of sensors disposed in an environment, the plurality of sensors configured to dynamically detect crop and environmental anomalies;
a processing system in communication with the plurality of sensors, the processing system configured to predict the crop and environmental anomalies; and
a mobile application configured to display the crop and environmental anomalies on a graphical user interface of a computing device.

12. The system of claim 8, wherein the plurality of sensors are configured to move throughout the environment via a sensor positioning system.

13. The system of claim 12, wherein the sensor positioning system is comprised of at least one of the following:
a cabling system;
a rail system;
a magnetic line system; or
a fixed post

14. The system of claim 13, wherein the sensor positioning system is further comprised of one or more UAV's configured to move at least one sensor throughout the environment.

15. The system of claim 14, wherein the plurality of sensors is comprised of at least one of the following:
a GNSS system;
an optical camera;
an RGBD camera;
a thermal camera;
a hyperspectral camera;
a humidity sensor;
a temperature sensor;
a pressure sensor;
a luminosity sensor;
a CO2 sensor; or
an audio sensor.

16. The system of claim 15, wherein the plurality of sensors transmits output data to a database.

17. The system of claim 16, wherein the database is in operable communication with an artificial intelligence engine configured to predict environmental and crop anomalies.

18. A modular smart sensor system, comprising:
a plurality of sensors disposed in an environment, the plurality of sensors configured to dynamically detect crop and environmental anomalies, wherein the plurality of sensors are provided on an insect trap having at least one adhesive surface to retain the insect;
a processing system in communication with the plurality of sensors, the processing system configured to predict the crop and environmental anomalies;
a machine learning engine to receive environmental information from a sensor array and compare, via a comparator, the received information with information stored in a database to identify the environmental information; and
a mobile application configured to display the crop and environmental anomalies on a graphical user interface of a computing device.

19. The system of claim 18, wherein an analytics dashboard is provided on a computing device to provide environmental analytics.

20. The system of claim 19, wherein the sensor array is in operable communication with a computational module provided in a housing of the insect trap to analyze the environment information received from the sensor array.

21. The system of claim 19, wherein a mobile application is used to collect optical, audio and video from the environment or insect trap communicating the data cloud-based system for environmental analytics.

22. The system of claim 19, where big data aggregator software is used to query and use to create machine learning models for the specific environmental information, including predictions and trends, resulting in a service in form of an API. (Bionetworks)
